(19) United States
(12) Patent Application Publication
(10) Pub. No.: US 2024/0000296 A1
(43) Pub. Date: Jan. 4, 2024

(54) CONVERGING AXES STEREOSCOPIC IMAGING SYSTEMS

(71) Applicant: INTUITIVE SURGICAL OPERATIONS, INC., Sunnyvale, CA (US)

(72) Inventor: David C. Shafer, Menlo Park, CA (US)

(21) Appl. No.: 18/253,908

(22) PCT Filed: Nov. 19, 2021
(86) PCT No.: PCT/US2021/060014

§ 371 Date: May 22, 2023

Related U.S. Application Data
(60) Provisional application No. 63/117,335, filed on Nov. 23, 2020.

(51) Int. Cl. A61B 1/00; A61B 1/05

(52) U.S. Cl. CPC A61B 1/00193; A61B 1/00096; A61B 1/051; A61B 1/00188

(57) ABSTRACT

A stereoscopic endoscope may comprise a first image capture sensor comprising a first surface and a second image capture sensor comprising a second surface. The endoscope also may comprise a first objective lens assembly to direct first light to the first surface. The first light extends along a first distal optical axis through the first objective lens assembly and extends along a first proximal optical axis after exiting. The first proximal optical axis intersects the first surface. The endoscope may also comprise a second objective lens assembly to direct second light to the second surface. The second light extends along a second distal optical axis through the second objective lens assembly and extends along a second proximal optical axis after exiting. The second proximal optical axis intersects the second surface. The first distal optical axis may be non-parallel to the second distal optical axis.

### CROSS-REFERENCE TO RELATED APPLICATIONS

This application claims the benefit of U.S. Provisional Application 63/117,335 filed Nov. 23, 2020, which is incorporated by reference herein in its entirety.

### FIELD

Examples described herein are related to stereoscopic imaging systems with converging optical axes.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments to reach a target tissue location. Minimally invasive medical tools include instruments such as therapeutic instruments, diagnostic instruments, imaging instruments, and surgical instruments. In some examples, a minimally invasive medical tool may be a stereo-imaging instrument, such as a stereoscopic endoscope, for generating three-dimensional images of anatomic areas within a patient anatomy. Stereo-imaging instruments may include a pair of objective lens assemblies for directing light to an image sensing system to generate a stereo pair of images.

### SUMMARY

The following presents a simplified summary of various examples described herein and is not intended to identify key or critical elements or to delineate the scope of the claims.

A stereoscopic endoscope may comprise a first image capture sensor comprising a first surface and a second image capture sensor comprising a second surface. The endoscope also may comprise a first objective lens assembly to direct first light to the first surface. The first light extends along a first distal optical axis through the first objective lens assembly and extends along a first proximal optical axis after exiting. The first proximal optical axis intersects the first surface. The endoscope may also comprise a second objective lens assembly to direct second light to the second surface. The second light extends along a second distal optical axis through the second objective lens assembly and extends along a second proximal optical axis after exiting. The second proximal optical axis intersects the second surface. The first distal optical axis may be non-parallel to the second distal optical axis.

In another example a method may include directing a first light along a first distal optical axis through a first objective lens assembly. After exiting the first objective lens assembly, the first light may be directed along a first proximal optical axis to a first surface of a first image capture sensor. The first proximal optical axis may be non-perpendicular to the first surface of the first image capture sensor. The method also includes directing a second light along a second distal optical axis through a second objective lens assembly. After exiting the second objective lens assembly, the second light may be directed along a second proximal optical axis to a second surface of a second image capture sensor. The first distal optical axis may be non-parallel to the second distal optical axis.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates a distal end of a stereoscopic imaging system according to some examples.

FIG. 2 is a schematic illustration of a stereoscopic imaging system including a pair of imaging assemblies with converging optical axes and sensor surfaces perpendicular to optical axes according to some examples.

FIG. 3 is a schematic illustration of a stereoscopic imaging system including a pair of imaging assemblies with converging optical axes and sensor surfaces non-perpendicular to optical axes according to some examples.

FIG. 4 is a schematic illustration of a stereoscopic imaging system including a pair of imaging assemblies with converging optical axes, optical elements for directing light, and sensor surfaces perpendicular to optical axes according to some examples.

FIG. 5A illustrates an optical element and image sensor according to some embodiments.

FIG. 5B illustrates an optical element and image sensor according to some embodiments.

FIG. 5C illustrates an optical element and tilted image sensor according to some embodiments.

FIG. 6 illustrates an exploded perspective view of the stereoscopic imaging system with movable components in the imaging assemblies according to some embodiments.

FIG. 7A illustrates a half portion of a stereoscopic imaging system including an optical element and a pair of image sensors according to some embodiments.

FIG. 7B illustrates a half portion of a stereoscopic imaging system including an optical element and a pair of image sensors according to some embodiments.

FIG. 8 is a chart illustrating the influence of optical assembly design and the relationship between object distance and sensor tilt.

FIG. 9 illustrates a portion of a stereoscopic imaging system including an optical element and adjustable image sensors according to some embodiments.

FIG. 10 is a flowchart illustrating a method of generating stereoscopic images, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The technology described herein provides stereoscopic imaging systems with converging optical axes that may allow for imaging sensors with large image capture surfaces to capture larger and/or higher resolution images. Stereoscopic imaging systems with converging optical axes described herein may also utilize entrance pupil distances that provide correct stereo vision geometry.

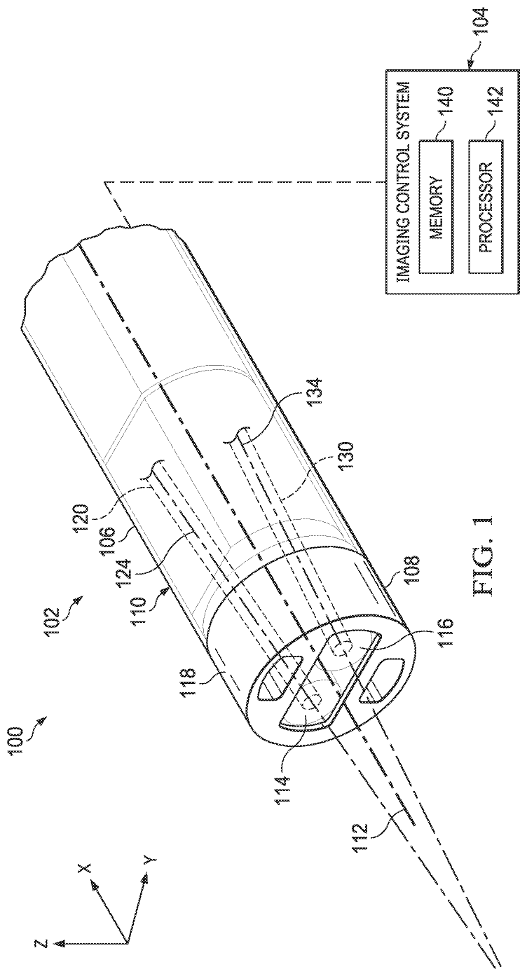

FIG. 1 illustrates a stereoscopic imaging system 100 that may be a stereoscopic endoscope system in some examples. The stereoscopic imaging system 100 may include an imaging instrument 102 coupled to an imaging control system 104. The imaging instrument 102 may be in an environment having a Cartesian coordinate system X, Y, Z. The imaging instrument 102 may include an elongate body 106 and an imaging device 108 that is coupled to a distal end 110 of the elongate body 106. A longitudinal axis 112 may extend through the imaging instrument 102. The elongate body 106 may be flexible or rigid, and the distal end 110 may be inserted into a patient anatomy to obtain stereoscopic images of anatomic tissue. In some examples, the patient anatomy may be a patient trachea, lung, colon, intestines, stomach, liver, kidneys and kidney calices, brain, heart, circulatory system including vasculature, and/or the like.

The imaging device 108 includes a right objective lens assembly 114 and a left objective lens assembly 116 inside of a housing 118. In the example of FIG. 1, the housing 118 may extend at least partially into a distal opening of the elongate body 106. In other examples, the housing 118 may extend over or abut to the distal end 110 of the elongate body 106. The right objective lens assembly 114 and the left objective lens assembly 116 may be arranged symmetrically about the longitudinal axis 112. Light 120 entering the right objective lens assembly 114 may extend along an optical axis 124 (e.g., a first distal optical axis) of the objective lens assembly 114. The light 120 may be centered about or symmetrical about the optical axis 124. Light 130 entering the left objective lens assembly 116 may extend along an optical axis 134 (e.g., a second distal optical axis) of the objective lens assembly 116. The light 130 may be centered about or symmetrical about the optical axis 134. As will be described below, the optical axes 124,134 may be non-parallel to the longitudinal axis 112 such that the optical axes 124, 134 converge distally of the imaging device 108 at a working distance from the distal end of the imaging device 108. A view target may be located at the working distance where the optical axes 124, 134 converge or at some closer or farther distance.

In some examples, the imaging instrument 102 may also include auxiliary systems such as illumination systems, cleaning systems, irrigation systems and/or other systems (not shown) to assist the function of the imaging device 108. In some examples, the imaging instrument 102 may also house cables, linkages, or other steering controls (not shown) to effectuate motion (e.g., pitch and yaw motion) of the distal end 110 of the elongate body 106.

The imaging control system 104 may include at least one memory 140 and at least one computer processor 142 for effecting control of imaging instrument 102, including recording image data, sending signals to and receiving information and/or electrical signals from the imaging assembly, operating an auxiliary system, moving the imaging device 108, and/or other functions of the imaging instrument 102. In some embodiments, the imaging control system 104 may be coupled to or be a component of a control system of a robot-assisted medical system. The imaging control system 104 may also include programmed instructions (e.g., a non-transitory machine-readable medium storing the instructions) to implement some or all of the methods described in accordance with aspects disclosed herein.

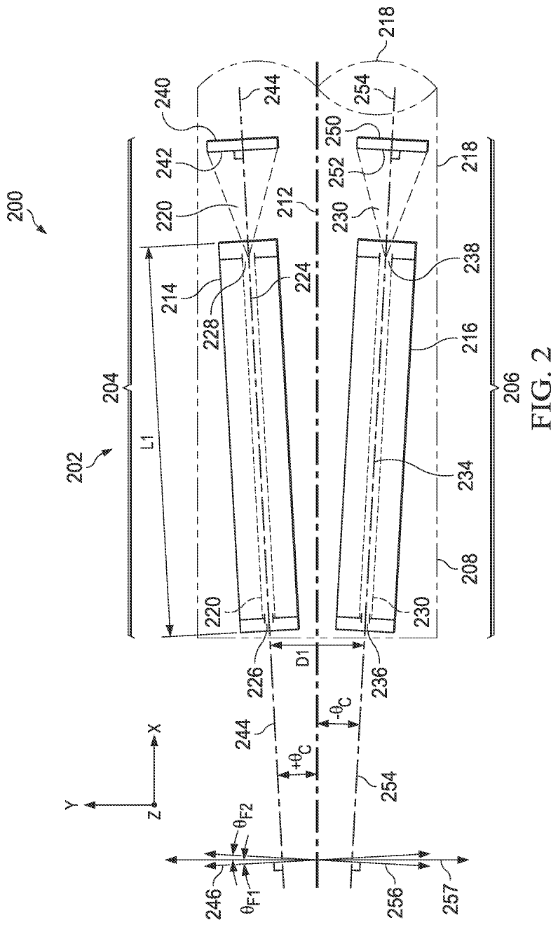

FIG. 2 provides a schematic illustration of a stereoscopic imaging system 200 (e.g., imaging system 100). The stereoscopic imaging system 200 includes an imaging instrument 202. The imaging instrument 202 may be in the environment having a Cartesian coordinate system X, Y, Z. The imaging instrument 202 may include an imaging device 208. A longitudinal axis 212 may extend through the imaging instrument 202. The imaging device 208 may include a right imaging assembly 204 comprising a right objective lens assembly 214 and a right image capture sensor 240 inside of a housing 218. The imaging device 208 also includes a left imaging assembly 206 comprising a left objective lens assembly 216 and a left image capture sensor 250 inside of the housing 218. The right objective lens assembly 214 and the left objective lens assembly 216 may be arranged symmetrically about the longitudinal axis 212. Light 220 entering the right objective lens assembly 214 may extend along an optical axis 224 (e.g., a first distal optical axis) of the objective lens assembly 214. The light 220 may be centered about or symmetrical about the optical axis 224. Light 230 entering the left objective lens assembly 216 may extend along an optical axis 234 (e.g., a second distal optical axis) of the objective lens assembly 216. The light 230 may be centered about or symmetrical about the optical axis 234. In this example, the optical axes 224, 234 are non-parallel to the longitudinal axis 212 such that the optical axes 224, 234 converge distally of the imaging device 208 and/or diverge proximally of the imaging device 208. In this example, the optical axis 224 is also non-parallel to the optical axis 234. The optical axis 224 may be tilted at a convergence angle +θ_{C} relative to the longitudinal axis 212, and the optical axis 234 may be tilted at a convergence angle −θ_{C} relative to the longitudinal axis 212. While the convergence angles θ_{C} of the optical axes 224, 234 are shown as being the same in FIG. 2, the convergence angles may be different in other examples. In some examples, the optical axes may converge, but the convergence angles relative to the longitudinal axis may be different.

The objective lens assembly 214 may include a single lens or may include a plurality of lenses, mirrors, prisms, and/or other optical elements to direct the light 220 along the optical axis 224 between an entrance pupil 226 at a distal end of the objective lens assembly 214 and an exit pupil 228 at a proximal end of the objective lens assembly 214. The objective lens assembly 216 may include a single lens or may include a plurality of lenses, minors, prisms, and/or other optical elements to direct the light 230 along the optical axis 234 between an entrance pupil 236 at a distal end of the objective lens assembly 216 and an exit pupil 238 at a proximal end of the objective lens assembly 216. An interpupillary distance D1 extends between the centers of the entrance pupils 226 and 236. To maintain an undistorted stereo disparity in the recorded stereo image pair, the ratio of the interpupillary distance D1 to the distance to viewed object, may be approximately the same as the ratio of the distance between the viewer's eyes to the distance to the stereo display. For some systems comprising a stereo display at close range to the viewer, the interpupillary distance may be between approximately 3.5 mm and 5. 5mm. For some systems comprising a stereo display but viewed from a greater distance, such as approximately 2 meters, the interpupillary distance may be smaller, such as between approximately 0.8 and 2.0 mm. If the entrance pupils are closer together than preferred, the disparity between the images in the stereo pair may be less than preferred and the viewer's sense of depth perception may be reduced. If, however, the distance between the entrance pupils is greater than preferred, the disparity is also greater, resulting in an exaggerated sense of depth perception and images that may be difficult to fuse and uncomfortable to watch.

In some examples, the objective lens assemblies 214, 216 may have a length L1 between approximately 20 mm and 25 mm. In some examples, the length L1 may be even smaller, for example 10 mm or smaller. In some examples, the length L1 may be longer. A diameter of imaging device 208 may depend on the size of the image sensors 240 and 250 and may be larger than the distance D1 between the entrance pupils 226 and 236. In some examples, the diameter of the imaging device 208 may range between 10 and 20 mm to accommodate high-resolution image sensors, while the distance D1 for comfortable stereo viewing may be between approximately 0.8 and 2.0 or 3.5 and 5.5 mm for displays that are 2 meters or 0.5 meters from the viewer, respectively. In some examples, the diameter of the imaging device 208 may range between approximately 10 and 20 mm for displays that are viewed from approximately 0.3 and 1.0 meters (e.g. distance from display to the viewer's eyes).

The right image capture sensor 240 includes a right image capture surface 242, and the left image capture sensor 250 includes a left image capture sensor surface 252. The light 220 exiting the exit pupil 228 may extend along an optical axis 244 (e.g., a first proximal optical axis) that intersects the image capture surface 242. The light 230 exiting the exit pupil 238 may extend along an optical axis 254 (e.g., a second proximal optical axis) that intersects the image capture surface 252.

In this example, the optical axis 244 may be approximately perpendicular to the image capture surface 242 and may be collinear with the optical axis 224. The optical axis 254 may be approximately perpendicular to the image capture surface 252 and may be collinear with the optical axis 234. A focal plane 246 of the imaging assembly 204 may be approximately perpendicular to the optical axis 224. A focal plane 256 of the imaging assembly 206 may be approximately perpendicular to the optical axis 234. As shown in FIG. 2, with this configuration of the imaging assemblies 204, 206, the focal planes 246, 256 may be slightly skewed or non-coplanar. In this example, the focal plane 246 may be rotated an angle θ_{F1} relative to a plane 257 that is perpendicular to the longitudinal axis 212. The angle θ_{F1} may have a magnitude that is the same or approximately the same magnitude as the angle θ_{C}. The focal plane 256 may be rotated an angle θ_{F2} relative to the plane 257. The angle θ_{F2} may have a magnitude that is the same or approximately the same magnitude as the angle θ_{C}. These non-coplanar focal planes may or may not cause viewer discomfort or inaccurate stereoscopic perception. Some scenarios (e.g., using higher resolution sensors, such as 4K or 8K sensors, or capturing images with a smaller depth of field), place high demands on the image alignment. Other scenarios (e.g., for lower resolution sensors or capturing image with a larger depth of field) may place lower demands on the image alignment. If the angle of convergence and the resulting non-coplanar focal plane cause viewer discomfort or inaccuracies, the focal planes 246, 256 may be adjusted to be substantially coplanar.

Adjusting the alignment of the focal planes 246, 256 to be substantially coplanar may be accomplished by arranging the objective lens assemblies 214, 216 so that the optical axes 224, 234 are parallel to each other and/or parallel to the longitudinal axis 212. However, given the constraints on the interpupillary distance D1 (e.g., approx. 3.5-5.5 mm) to maintain acceptable depth fidelity, parallel optical axes 224, 234 may bring the exit pupils 228, 238 closer together, which may leave insufficient space for the image capture sensors 240, 250 or require smaller image capture sensors.

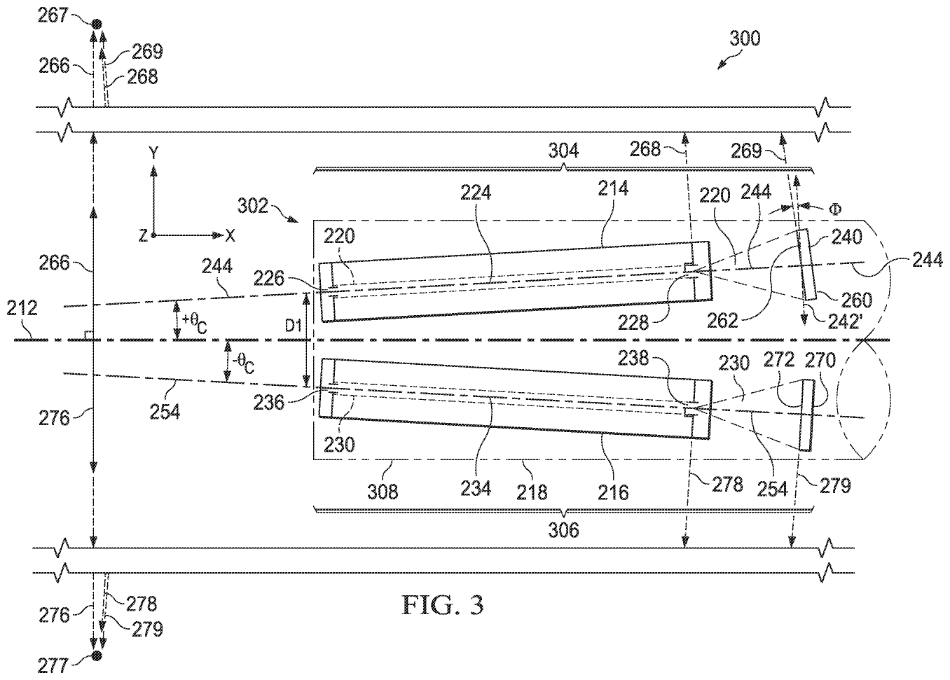

Alternatively, the misalignment of the focal planes 246, 256 may be compensated for as shown in the example of FIG. 3. FIG. 3 provides a schematic illustration of a stereoscopic imaging system 300. Components common to the stereoscopic imaging system 200 are indicated with the same reference numerals. The stereoscopic imaging system 300 includes an imaging instrument 302. The imaging instrument 302 may include an imaging device 308. A longitudinal axis 212 may extend through the imaging instrument 302. The imaging device 308 may include a right imaging assembly 304 comprising the right objective lens assembly 214 and a right image capture sensor 260 inside of the housing 218. The imaging device 308 also includes a left imaging assembly 306 comprising the left objective lens assembly 216 and a left image capture sensor 270 inside of the housing 218.

The right image capture sensor 260 may include a right image capture surface 262, and the left image capture sensor 270 may include a left image capture sensor surface 272. The light 220 exiting the exit pupil 228 may extend along an optical axis 244 (e.g., a first proximal optical axis) that intersects the image capture surface 262. The light 230 exiting the exit pupil 238 may extend along an optical axis 254 (e.g., a second proximal optical axis) that intersects the image capture surface 272.

The right image capture surface 262 may be tilted relative to the longitudinal axis 212 (e.g., rotated slightly counter-clockwise as compared to the right image capture surface 242 of system 200 that is perpendicular to the longitudinal axis 212) so that the optical axis 244 is non-perpendicular to the surface 262. The left image capture surface 272 may similarly be tilted relative to the longitudinal axis 212 (e.g., rotated slightly clockwise as compared to the left image capture surface 252 of system 200) so that the optical axis 274 is non-perpendicular to the surface 272. As a consequence of the tilted image capture surface 262, a focal plane 266 of the right imaging assembly 304 becomes rotated clockwise with respect to the longitudinal axis 212 (and clockwise as compared to the focal plane 246 of system 200). As a consequence of the tilted image capture surface 272, a focal plane 276 of the left imaging assembly 306 becomes rotated counter-clockwise with respect to the longitudinal axis 212 (and counter-clockwise as compared to the focal plane 256 of system 200). As shown in FIG. 3, with the optical axis 244 intersecting at a non-perpendicular angle to the surface 262 and with the optical axis 254 intersecting at a non-perpendicular angle to the surface 272, the focal planes 266, 276 may become aligned and coplanar or coincident, which may reduce stereo image distortion in some scenarios as compared to the system 200 of FIG. 2. The focal planes 266, 276 may be approximately perpendicular to the longitudinal axis 212. In alternative examples (and as will be described in further detail below), rather than changing the angle of the image capture surfaces, one or more faces of a prism located between an objective lens assembly and a corresponding image capture surface may be adjusted by an angle needed to cause the focal planes to become coincident.

To achieve the coplanar focal planes 266, 276, the right image capture surface 262 may be rotated an angle of rotation φ from the image capture surface 242 perpendicular to the optical axis 224. To more clearly show the angle φ, a plane 242′ that includes the surface 242 and an image capture plane 269 including the surface 262 are illustrated in FIG. 3. The relationship between the angle of rotation φ and the angle θ_{F1} of the tilt of focal plane 246 may be related as described in the equation:

tan φ=m·tan θ_{F1},

where m is the magnification of the objective lens assembly. The sign of the magnification m is negative if the right objective lens assembly 214 includes a simple lens that causes inversion of the image. In an example in which the magnification of the right objective lens assembly 214 is small (e.g. approximately 0.05), the tilt of the image capture surface 262 (the angle φ) is also small (e.g. less than approximately 1°). Applications of the Scheimpflug principle may be used to correct the focus of an optical system when the image plane, the lens plane, and the focal plane are not parallel. The Scheimpflug principle describes the geometric relationship between a plane of focus, a lens plane, and an image plane of an optical system when the lens plane is not parallel to the image plane. With the tilt of the image capture plane 269 determined, the focal plane 266, the image plane 269, and a Z direction plane 268 through the lens plane at the exit pupil 228 may intersect at an intersection line 267 extending in the Z direction, perpendicular to the longitudinal axis 212.

The rotation of the left image capture surface 272 relative to the image capture surface 252 may likewise be determined based on the above recited relationship with the angle of the tilt of focal plane (e.g. angle θ_{F2}). The focal plane 276, a plane 279 through the rotated image capture surface 272, and a Z direction plane 278 through the plane of the exit pupil 238 may intersect at an intersection line 277 extending in the Z direction, perpendicular to the longitudinal axis 212. With the planes 266, 268, 269 intersecting at the line 267 and the planes 276, 278, 279 intersecting at the line 277, the focal planes 266, 276 may be aligned and co-planar.

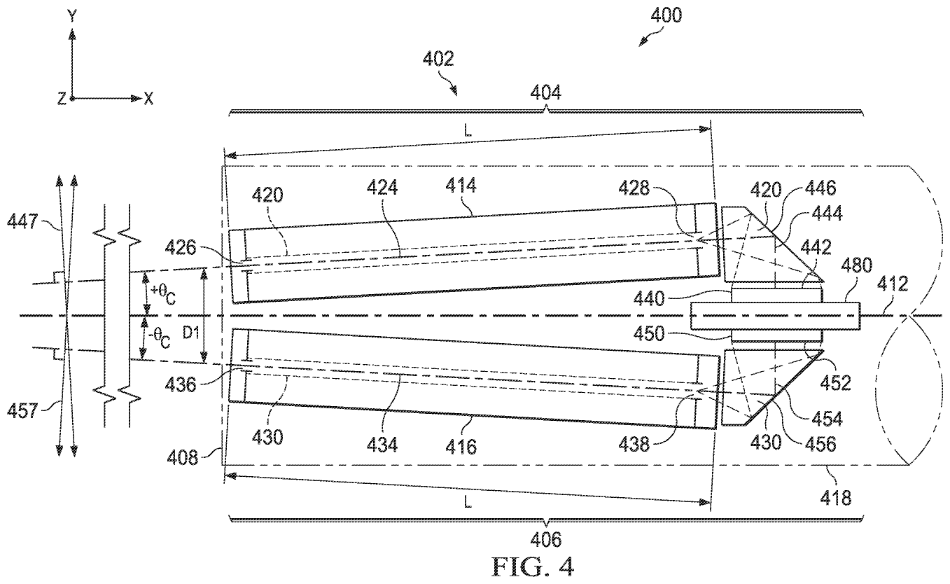

In the examples of FIGS. 2 and 3, the length and/or area of the image capture sensors may be limited by the diameter of the imaging instrument because the sensors are perpendicular or nearly perpendicular to the longitudinal axis of the imaging instrument. In alternative examples, sensors may be arranged parallel to or nearly parallel to the longitudinal axis of the imaging instrument, allowing for longer sensor lengths and/or larger areas. FIG. 4 provides a schematic illustration of a stereoscopic imaging system 400 (e.g., imaging system 100). The stereoscopic imaging system 400 includes an imaging instrument 402. The imaging instrument 402 may be in an environment having a Cartesian coordinate system X, Y, Z. The imaging instrument 402 may include an imaging device 408. A longitudinal axis 412 may extend through the imaging instrument 402. The imaging device 408 may include a right imaging assembly 404 comprising a right objective lens assembly 414, a right image capture sensor 440, and a right optical element 446 between the lens assembly 414 and the image capture sensor 440, inside of a housing 418. The imaging device 408 also includes a left imaging assembly 406 comprising a left objective lens assembly 416, a left image capture sensor 450, and a left optical element 456 between the lens assembly 416 and the image capture sensor 450, inside of a housing 418. The right objective lens assembly 414 and the left objective lens assembly 416 may be arranged symmetrically about the longitudinal axis 412. Light 420 entering the right objective lens assembly 414 may extend along an optical axis 424 (e.g., a first distal optical axis) of the objective lens assembly 414. The light 420 may be centered about or symmetrical about the optical axis 424. Light 430 entering the left objective lens assembly 416 may extend along an optical axis 434 (e.g., a second distal optical axis) of the objective lens assembly 416. The light 430 may be centered about or symmetrical about the optical axis 434. In this example, the optical axes 424, 434 are non-parallel to the longitudinal axis 412 such that the optical axes 424, 434 converge distally of the imaging device 408 and/or diverge proximally of the imaging device 408.

The objective lens assembly 414 may include a single lens or may include a plurality of lenses, mirrors, prisms, and/or other optical elements to direct the light 420 along the optical axis 424 between an entrance pupil 426 at a distal end of the objective lens assembly 414 and an exit pupil 428 at a proximal end of the objective lens assembly 414. The objective lens assembly 416 may include a single lens or may include a plurality of lenses, minors, prisms, and/or other optical elements to direct the light 430 along the optical axis 434 between an entrance pupil 436 at a distal end of the objective lens assembly 416 and an exit pupil 438 at a proximal end of the objective lens assembly 416. An interpupillary distance D1 extends between the centers of the entrance pupils 426 and 436. To maintain acceptable depth fidelity in the recorded stereo images, the distance D1 may be between approximately 3.5 mm and 5.5 mm.

In this example, the right image capture sensor 440 may be mounted to a support 480 and may include a right image capture surface 442 that extends approximately parallel to the longitudinal axis 412. The optical element 446 may be a prism extending between the objective lens assembly 414 and the right image capture sensor 440. In some examples, the sensor 440 may be coupled to the optical element 446. The left image capture sensor 450 may be mounted to an opposite side of the support 480 and may include a left image capture surface 452 that extends approximately parallel to the longitudinal axis 412. In this example, the optical element 456 may be a prism extending between the objective lens assembly 416 and the right image capture sensor 450. The light 420 exiting the exit pupil 428 may engage with the optical element 446, which may redirect the light 420 along an optical axis 444 (e.g., a first proximal optical axis) that intersects the image capture surface 442. The light 430 exiting the exit pupil 438 may engage with the optical element 456, which may redirect the light 430 along an optical axis 454 (e.g., a second proximal optical axis) that intersects the image capture surface 452.

In this example, the optical axis 444 may be approximately perpendicular to the right image capture surface 442. The optical axis 454 may be approximately perpendicular to the left image capture surface 452. A focal plane 447 of the imaging assembly 404 is approximately perpendicular to the optical axis 424. A focal plane 457 of the imaging assembly 406 is approximately perpendicular to the optical axis 434. With this configuration of the imaging assemblies 404, 406, the focal planes 447, 457 may be skewed or non-coplanar. As previously explained, non-coplanar focal planes may result in stereo image distortion in some scenarios.

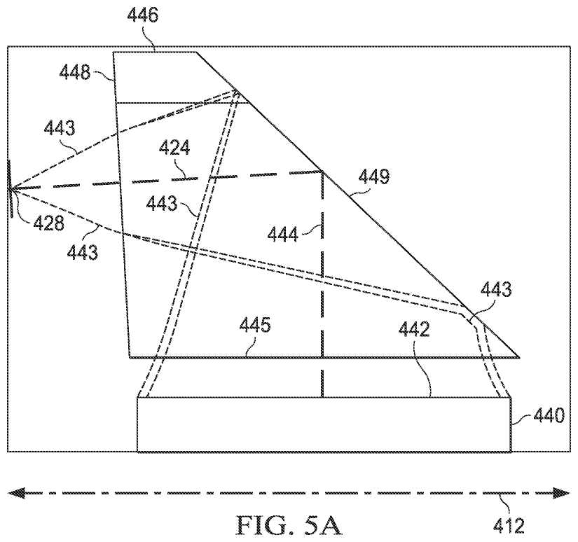

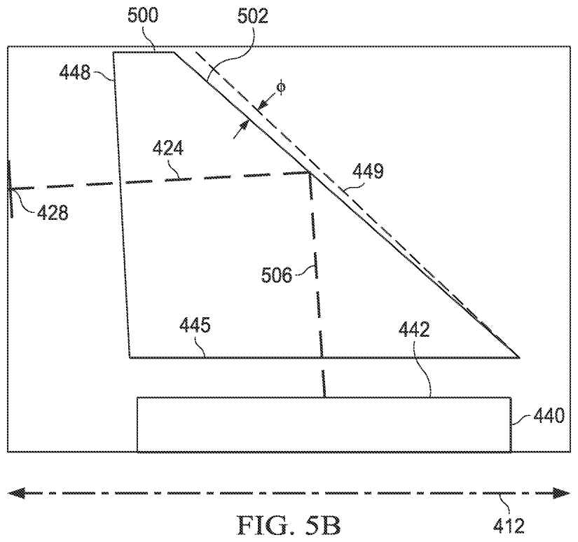

FIG. 5A illustrates an example of the optical element 446 and right image capture sensor 440. The optical element 446 includes an optical element entry face 448, a reflection face 449 and an exit face 445. The light 420 exiting the exit pupil 428 enters the entry face 448 extending along the optical axis 424. The entry face 448 may be sized to receive the external edges 443 of the light 420. The light 420 may reflect off of the reflection face 449 at an angle that depends on the angle of the reflection face 449. For example, assuming that the optical axis 424 is parallel to the longitudinal axis of the imaging instrument (e.g., longitudinal axis 412 in FIG. 4), the reflection face 449 may be angled at 45° relative to the longitudinal axis of the instrument. The light 420 reflecting off of the reflection face 449 may hit the surface 442 and be captured by the image capture sensor 440 sized to receive the external edges 443 of the light 420. In examples such as FIGS. 4 and 5A where the optical axis 424 is non-parallel to the longitudinal axis 412, the reflection face 449 may rotated counterclockwise from 45° relative to the longitudinal axis 412 by an additional angle of θ_{C}/2. Thus, the reflection face 449 may be at an angle of 45° minus θ_{C}/2, relative to the longitudinal axis 412. The size, shape, and/or configuration of the optical element 446 may be determined by factoring in (e.g., optimizing) design parameters including a field of view of the objective lens assembly 414; the distance from the entrance pupils 426, 436 at which the optical axes 424, 434 converge; the interpupillary distance D1; a convergence angle θ_{C}; an image diameter; a length L of the objective lens assembly 414; the sensor loft distance between the longitudinal axis 412 and the surface 442; the inner diameter of the housing 418; a distance between the exit pupil 428 and the optical element entry face 448; a glass index for the optical element; an f-number for the imaging assembly 404; a target aspect ratio; an acceptable distortion threshold level; and/or a border distance between an edge of the optical element 446 and all incident light 420. In some examples, the angle of the reflection face 449, the angle of the entry face 448, and/or the angle of the exit face 445 may be adjusted by an angle needed to cause the focal planes of the right and left imaging assemblies to become coincident.

In some examples to adjust for the distortion due to the non-coplanar focal planes 447, 457 of the system 400, an optical element 500 may replace the optical element 446 in the stereoscopic imaging system 400, as shown in FIG. 5B. The optical element 500 may be a prism extending between the objective lens assembly 414 and the right image capture sensor 440. In this example, the optical element 500 includes a reflection surface 502 that is adjusted counterclockwise, relative to the reflection surface 449, by a magnitude of φ/2. As described above, φ may be related to the tilt of the focal plane 447 (relative to the plane perpendicular to the longitudinal axis 412) by the equation:

tan φ=m·tan[angle of focal plane tilt].

The light 420 exiting the exit pupil 428 enters the entry face 448 extending along the optical axis 424. The light may reflect off of the reflection face 502 and may be redirected along an optical axis 506 (e.g., a first proximal optical axis) that intersects the image capture surface 442 at a non-perpendicular angle. The adjusted reflection face 502 may cause the focal plane 447 to become approximately perpendicular to the longitudinal axis 412, and a similar adjustment to the optical element 456 will tilt the focal plane 457 to become approximately perpendicular to the longitudinal axis, thus causing both focal planes 447, 457 to become coincident.

In some examples to adjust for the distortion due to the non-coplanar focal planes 447, 457 of the system 400, the sensor surfaces may be tilted so that the proximal optical axis is not perpendicular to the sensor surface as shown in FIG. 5C. An optical element 520 may replace the optical element 446 in the stereoscopic imaging system 400. The optical element 520 may be a prism extending between the objective lens assembly 414 and the right image capture sensor 440. In this example, the right image capture sensor 440 is tilted, by a magnitude of φ clockwise and relative to the longitudinal axis 412. As described above, φ may be related to the tilt of the focal plane 447 (relative to the plane perpendicular to the longitudinal axis 412) by the equation:

tan φ=m·tan[angle of focal plane tilt].

The light 420 exiting the exit pupil 428 enters the entry face 448 extending along the optical axis 424. The light may reflect off of the reflection face 449 and may be redirected along the optical axis 444 (e.g., a first proximal optical axis) that intersects the image capture surface 442 at a non-perpendicular angle. In this example an exit surface 522 of the optical element may be parallel to the tilted image capture surface 452. In this example, the right image capture sensor 440 may be mounted to a support 524 which may support the sensor 440 in the tilted pose. The left image capture sensor 450 may be mounted to an opposite side of the support 524 which may support the sensor 470 in the tilted pose. As a consequence of the tilted image capture surface 442, the focal plane 447 becomes rotated clockwise. A similar adjustment to the image capture surface 452 will rotate the focal plane 457 counter-clockwise. Thus, the focal planes 447, 457 may become aligned and coplanar or coincident which may reduce stereo image distortion in some scenarios as compared to the system 400 of FIG. 4. The focal planes 467, 477 may be approximately perpendicular to the longitudinal axis 412. In various examples, the tilt angle of the image capture sensor 440 and/or the angle of the reflection face of the optical element may be adjustable via a manually controlled or electronically controlled actuator.

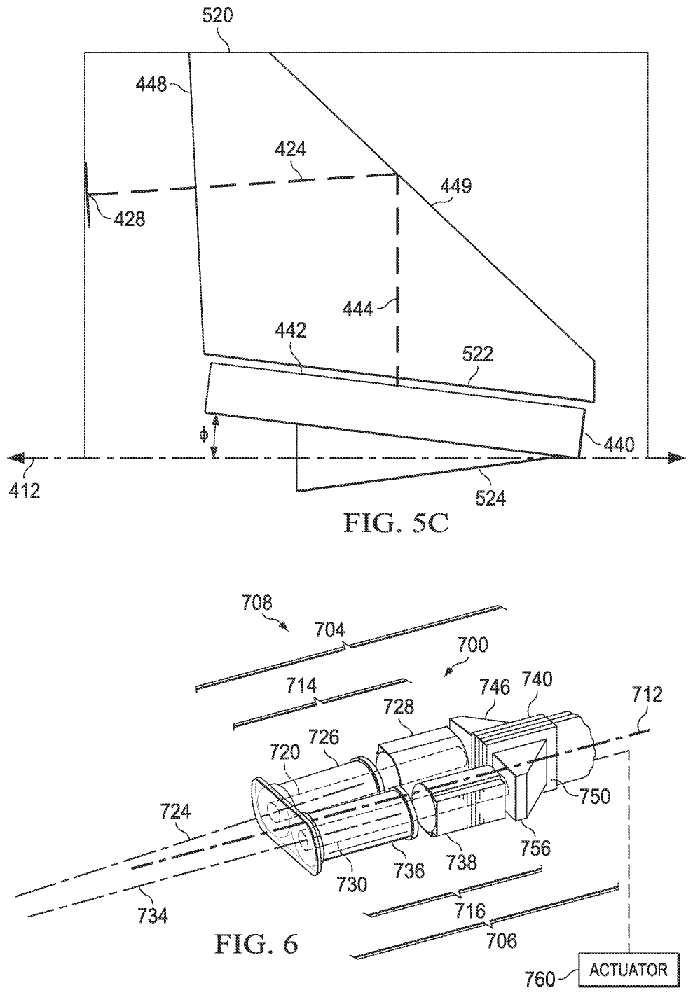

FIG. 6 is an exploded perspective view of a stereoscopic imaging system 700 (e.g., imaging system 100). In this example, the system may be substantially similar to the imaging system 400 but may have adjustable components in the objective lens assemblies. The stereoscopic imaging system 700 includes an imaging device 708 extending along a longitudinal axis 712. The imaging device 708 may include a right imaging assembly 704 comprising a right objective lens assembly 714, a right image capture sensor 740, and a right optical element 746. The imaging device 708 may also include a left imaging assembly 706 comprising a left objective lens assembly 716, a left image capture sensor 750, and a left optical element 756. The right objective lens assembly 714 and the left objective lens assembly 716 may be arranged symmetrically about the longitudinal axis 712. Light 720 entering the right objective lens assembly 714 may extend along an optical axis 724 (e.g., a first distal optical axis) of the objective lens assembly 714. The light 720 may be centered about or symmetrical about the optical axis 724. Light 730 entering the left objective lens assembly 716 may extend along an optical axis 734 (e.g., a second distal optical axis) of the objective lens assembly 716. The light 730 may be centered about or symmetrical about the optical axis 734. In this example, the optical axes 724, 734 may be non-parallel to the longitudinal axis 712 such that the optical axes 724, 734 converge distally of the imaging device 708.

The right objective lens assembly 714 may include a lens component 726 co-axial with a lens component 728. Either or both of the lens components 726, 728 may be movable to adjust the focus of the right objective lens assembly 714. The movement of any of the lens components 726, 728 may be actuated by an actuator system 760 which may be, for example, a motor. In some examples, the actuator system 760 and the focusing of the right objective lens assembly may be controlled by control signals received from the image control system 104. The left objective lens assembly 716 may include a lens component 736 co-axial with a lens component 738. Either or both of the lens components 736, 738 may be movable to adjust the focus of the right objective lens assembly 716. The movement of any of the lens components 736, 738 may be actuated by the actuator system 760. The actuator system 760, the focusing of the right objective lens assembly 714, and/or the focusing of the left objective lens assembly 716 may be controlled by control signals received from the image control system 104. The right objective lens assembly 714 may be focused independently of or in coordination with the left objective lens assembly 716. In some examples, separate actuators may control independent movement of the objective lens assemblies 714, 716. The separate actuators may be synchronized to provide minor-image motion (about the longitudinal axis) of the objective lens assemblies when the optical axes of the objective lens assemblies are not parallel.

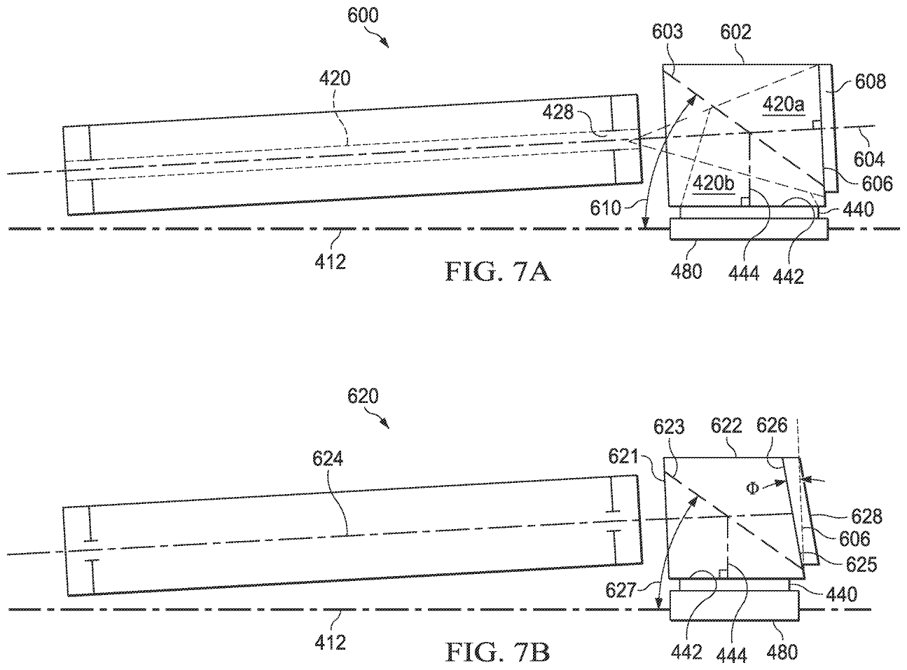

FIG. 7A provides a schematic illustration of a portion of a stereoscopic imaging system 600 (e.g., imaging system 100). The stereoscopic imaging system 600 may be substantially similar to the imaging system 400 but may have a different optical element 602. In this example, the optical element 602 may include a beam splitter 603 that splits light 420 causing a light portion 420a to be directed along an optical axis 604 to a sensor surface 606 of an image capture sensor 608 and a light portion 420b to be directed along the optical axis 444 toward the surface 442 of the image capture sensor 440. The image capture sensor 608 may be mounted directly to the optical element 602 or may be supported within the imaging system 600 by another support member. The image capture sensor 440 may be parallel to the longitudinal axis 412. In this example, the left optical element 456 (shown in FIG. 4) may also be replaced with an optical element having a beam splitter. As shown in FIG. 7A, the sensor surface 606 may be perpendicular to the optical axis 604, and the sensor surface 442 may be perpendicular to the optical axis 444 (and parallel to the longitudinal axis 412). In this example, the beam splitter 603 may rotated counterclockwise from 45° relative to the longitudinal axis 412 by an additional angle of θ_{C}/2, where θ_{C} is the convergence angle between the optical axis 604 and the longitudinal axis 412. Thus, the beam splitter 603 may be oriented at an angle 610, relative to the longitudinal axis 412, of 45° minus θ_{C}/2. The beam splitter 603 may split the incoming light into different wavelength bands and/or based on the sensor technology of the receiving sensors. For example, the beam splitter may direct a portion of the incoming light toward an infrared sensor and may direct another portion of the incoming light toward an ultraviolet sensor. As another example, the beam splitter may direct visible light toward one sensor and infrared or near infrared light (e.g., fluorescence emission light) to another sensor. As yet another example, the beam splitter may direct some visible light bands (e.g., red and blue bands) toward one sensor and other visible light bands (e.g., green bands) toward another sensor.

FIG. 7B provides a schematic illustration of a portion of a stereoscopic imaging system 620 (e.g., imaging system 100) with an optical element 622 that adjusts for the distortion due to the non-coplanar focal planes 447 of the system 400. The stereoscopic imaging system 620 may be substantially similar to the imaging system 400 but may have a different optical element 622. In this example, the optical element 602 may include a beam splitter 623 that splits light 420 causing a portion to be directed along an optical axis 624 to a sensor surface 626 of an image capture sensor 608 and a portion to be directed along the optical axis 444 toward the surface 442 of the image capture sensor 440. The image capture sensor 440 may be parallel to the longitudinal axis 412. In this example, the left optical element 456 (shown in FIG. 4) may also be replaced with an optical element having a beam splitter and geometry that incorporates the offset angle needed to rotate the focal plane 447 into alignment. As shown in FIG. 7B, the optical element 622 may include a proximal face 621 that is perpendicular to the optical axis 604 and a distal face 625 to which the sensor surface 626 may be coupled. The distal face 625 is offset counterclockwise from being perpendicular to the optical axis 604 by an angle φ. Likewise, the coupled sensor surface 626 may be tilted counterclockwise by the angle φ relative to the sensor surface 606 (as shown in FIG. 7A, which is perpendicular to the optical axis 604). Thus, the optical axis 624 (e.g., a first proximal optical axis) intersects the image capture surface 626 at a non-perpendicular angle. The sensor surface 442 may be perpendicular to the optical axis 444 (and parallel to the longitudinal axis 412). In this example, the beam splitter 623 may be rotated counterclockwise from 45°, relative to the longitudinal axis 412, by an additional angle of θ_{C}/2+φ/2. Thus, the beam splitter 623 may be oriented at an angle 627, relative to the longitudinal axis 412, of 45° minus (θ_{C}/2+φ/2). As a consequence of the tilted image capture surface 626 and the adjusted beam splitter 623, the focal plane 447 becomes rotated clockwise. A similar adjustment to the image capture surface 452 will rotate the focal plane 457 counter-clockwise. Thus, the focal planes 447, 457 may become aligned and coplanar or coincident which may reduce stereo image distortion in some scenarios as compared to the system 400 of FIG. 4. The focal planes 467, 477 may be approximately perpendicular to the longitudinal axis 412. With this example, the angle φ may be precisely controlled by the manufacture of the optical element and little or no additional compensation needed to the endoscope.

The beam splitter 623 may split the incoming light into different wavelength bands and/or based on the sensor technology of the receiving sensors. For example, the beam splitter may direct a portion of the incoming light toward an infrared sensor and may direct another portion of the incoming light toward an ultraviolet sensor. As another example, the beam splitter may direct visible light toward one sensor and infrared or near infrared light (e.g., fluorescence emission light) to another sensor. As yet another example, the beam splitter may direct some visible light bands (e.g., red and blue bands) toward one sensor and other visible light bands (e.g., green bands) toward another sensor.

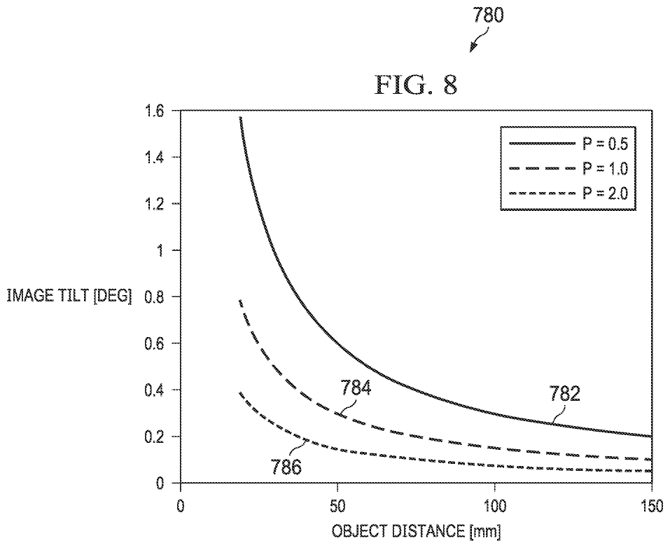

As an object distance between the entrance pupil 426 and the focal plane 447 varies, the tilt of the image capture sensor surface(s) to compensate for the fixed converging angle (e.g. θ_{C} may change). The change in the tilt of the image capture sensor surface may become large as the object distance decreases (i.e., when the entrance pupil is relatively close to the object of focus). The tilt of the image capture sensor surface may also depend on the asymmetry of the objective lens assembly. Because the Scheimpflug condition (where the object and image capture planes intersect at the lens plane) may apply primarily or only for thin lenses which are symmetric, the asymmetric lens of a typical endoscope may influence the tilt of the image capture sensor surface. The effect of the objective lens assembly asymmetry may be captured by a pupil magnification parameter P. FIG. 8 provides a chart 780 illustrating the tilt of the image capture sensor (Image Tilt [deg]) needed as a function of object distance (Object Distance [mm]) with a fixed convergence angle θ_{C} of, for example, 5°. A curve 782 illustrates a relationship between object distance and sensor tilt for a lens design with a pupil magnification of P=0.5. A curve 784 illustrates a relationship between object distance and sensor tilt for a lens design with a pupil magnification of P=1.0. A curve 786 illustrates a relationship between object distance and sensor tilt for a lens design with a pupil magnification of P=2.0. As illustrated by the curves 782-786, the tilt of the image capture sensor increases as the object distance decreases. The curves 782-786 also illustrate that for a given object distance, the tilt of the image capture sensor is greater at smaller pupil magnifications.

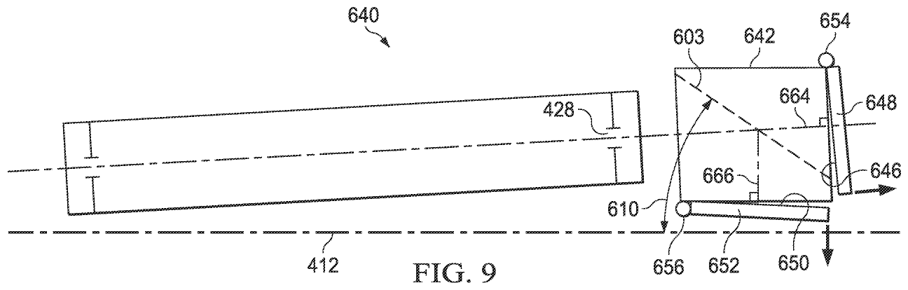

If the accuracy needed for a particular endoscope is high enough to require adjustment, the tilt of the image capture sensors may be adjusted. FIG. 9 provides a schematic illustration of a portion of a stereoscopic imaging system 640 (e.g., imaging system 100) with the optical element 642. In this example, the optical element 642 may include a beam splitter 643 that splits light 420 causing a portion to be directed along an optical axis 664 to a sensor surface 646 of an image capture sensor 648 and a portion to be directed along the optical axis 666 toward the surface 652 of an image capture sensor 650. In this example, the beam splitter 643 may be rotated counterclockwise from 45° relative to the longitudinal axis 412 by an additional angle of θ_{C}/2. Thus, the beam splitter 603 may be oriented at an angle, relative to the longitudinal axis 412, of 45° minus θ_{C}/2. A hinge 654 or other type of flexure device may couple the image capture sensor 648 to the optical element 642, and hinge 656 or other type of flexure device may couple the image capture sensor 650 to the optical element 642. One or more actuators (e.g., motors) coupled to the hinges 656, 654 may be activated to rotate the image capture sensors relative to the optical element and thus tilt the surfaces 646, 652. The actuation may be based on a user input, a position of the focus adjustment of the objective lens assembly, eye tracking, and/or image analysis.

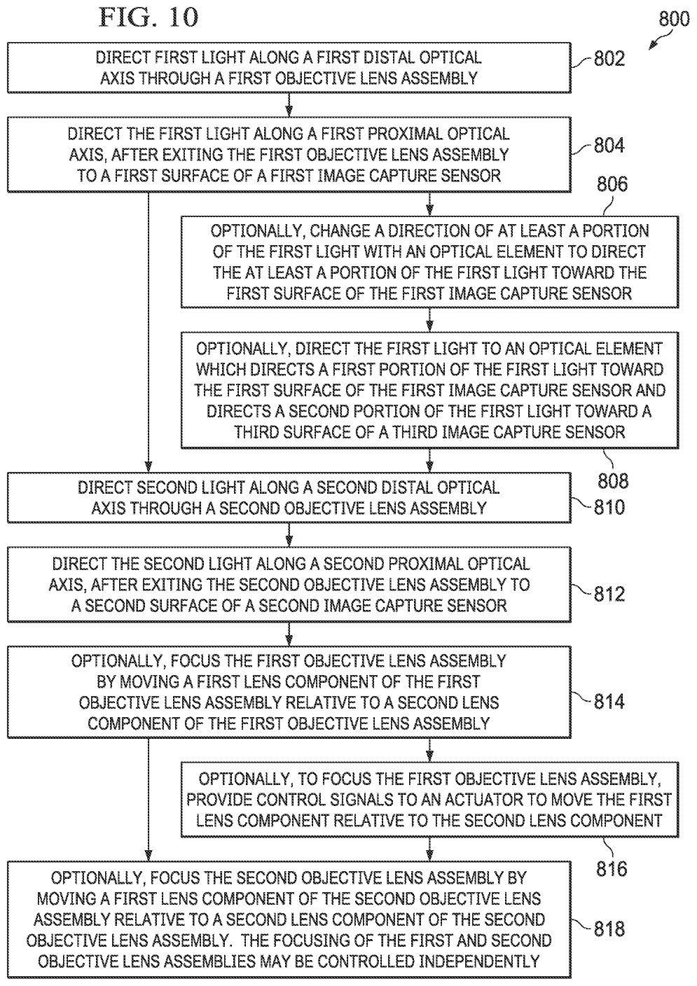

FIG. 10 is a flowchart illustrating an example method 800 for operating a stereoscopic imaging system, including any of those previously described. The method 800 is illustrated as a set of operations or processes 802 through 818. The processes illustrated in FIG. 10 may be performed in a different order than the order shown in FIG. 10, and one or more of the illustrated processes might not be performed in some embodiments of method 800. Additionally, one or more processes that are not expressly illustrated in FIG. 10 may be included before, after, in between, or as part of the illustrated processes. In some embodiments, one or more of the processes of method 800 may be implemented, at least in part, in the form of executable code stored on non-transitory, tangible, machine-readable media that when run by one or more processors (e.g., the processors of a control system) may cause the one or more processors to perform one or more of the processes.

At a process 802, first light (e.g., light 120, 220, 420, 720) may be directed along a first distal optical axis (e.g., optical axis 124, 224, 424, 724) through a first objective lens assembly (e.g., objective lens assembly 114, 214, 414, 714).

At a process 804, the first light may be directed along a first proximal optical axis (e.g., optical axis 244, 444, 604), after exiting the first objective lens assembly, to a first surface (e.g., surface 242, 262, 442, 462, 606) of a first image capture sensor (e.g., image capture sensor 240, 260, 440, 460, 608, 740). The first proximal optical axis may be non-perpendicular to the first surface of the first image capture sensor in some examples.

At a process 806, optionally, a direction of at least a portion of the first light may be changed with an optical element to direct the at least a portion of the first light toward the first surface of the first image capture sensor.

At a process 808, optionally, the first light may be directed to an optical element which directs a first portion of the first light toward the first surface of the first image capture sensor and directs a second portion of the first light toward a third surface of a third image capture sensor.

At a process 810, second light (e.g., light 130, 230, 430, 730) may be directed along a second distal optical axis (e.g., optical axis 134, 234, 434, 734) through a second objective lens assembly (e.g., objective lens assembly 116, 216, 416, 716). The first distal optical axis may be non-parallel to the second distal optical axis.

At a process 812, the second light may be directed along a second proximal optical axis (e.g., optical axis 254, 454), after exiting the second objective lens assembly, to a second surface (e.g. surface 252, 272, 452, 472) of a second image capture sensor (e.g., image capture sensor 250, 270, 450, 470, 750). The second proximal optical axis may be non-perpendicular to the second surface of the second image capture sensor in some examples.

At a process 814, optionally the first objective lens assembly may be focused by moving a first lens component of the first objective lens assembly relative to a second lens component of the first objective lens assembly.

At a process 816, optionally the first objective lens assembly may be focused by providing control signals to an actuator to move the first lens component relative to the second lens component.

At a process 818, optionally the second objective lens assembly may be focused by moving a first lens component of the second objective lens assembly relative to a second lens component of the second objective lens assembly. The focusing of the first and second objective lens assemblies may be controlled independently.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions. Not all the illustrated processes may be performed in all embodiments of the disclosed methods. Additionally, one or more processes that are not expressly illustrated in may be included before, after, in between, or as part of the illustrated processes. In some embodiments, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some embodiments are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in embodiments of this disclosure may be implemented in software to execute on a processor of a computer system such as control processing system. When implemented in software, the elements of the embodiments of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A stereoscopic endoscope comprising:
a first image capture sensor comprising a first surface;
a second image capture sensor comprising a second surface;
a first objective lens assembly configured to direct first light to the first surface of the first image capture sensor, wherein the first light extends along a first distal optical axis through the first objective lens assembly and wherein at least a portion of the first light extends along a first proximal optical axis after exiting the first objective lens assembly, wherein the first proximal optical axis intersects the first surface;
a second objective lens assembly configured to direct second light to the second surface of the second image capture sensor, wherein the second light extends along a second distal optical axis through the second objective lens assembly and extends along a second proximal optical axis after exiting the second objective lens assembly, wherein the second proximal optical axis intersects the second surface;
an optical element configured to receive the first light from the first objective lens assembly and direct the at least a portion of the first light along the first proximal optical axis, toward the first surface of the first image capture sensor,
wherein the first distal optical axis is non-parallel to the second distal optical axis and wherein the first proximal optical axis is non-perpendicular to the first surface of the first image capture sensor.

2. The stereoscopic endoscope of claim 1 wherein the second proximal optical axis is non-perpendicular to the second surface of the second image capture sensor.

3. The stereoscopic endoscope of claim 1 wherein a distance between an entrance pupil of the first objective lens assembly and an entrance pupil of the second objective lens assembly is between approximately 3.5 and 5.5 mm.

4. The stereoscopic endoscope of claim 1 wherein the first objective lens assembly has a length of approximately 25 mm.

5. (canceled)

6. The stereoscopic endoscope of claim 1 wherein the optical element includes a prism.

7. The stereoscopic endoscope of claim 6 wherein the prism includes a reflection face configured to reflect the at least the portion of the first light along the first proximal optical axis and toward the first surface of the first image capture sensor.

8. The stereoscopic endoscope of claim 1 further comprising:
a third image capture sensor comprising a third surface, wherein the optical element is configured to direct a first portion of the first light toward the first surface of the first image capture sensor and to direct a second portion of the first light toward the third surface of the third image capture sensor.

9. The stereoscopic endoscope of claim 8 wherein the optical element includes a beam splitter.

10. The stereoscopic endoscope of claim 8 further comprising a flexure device between the third image capture sensor and the optical element.

11. The stereoscopic endoscope of claim 10 further comprising an actuator configured to activate the flexure device to move the third image capture sensor relative to the optical element.

12. The stereoscopic endoscope of claim 8 wherein optical element includes a distal face and a proximal face, wherein the distal and proximal faces are non-parallel.

13. The stereoscopic endoscope of claim 1 wherein the first objective lens assembly includes a first lens component and a second lens component co-axial with the first lens component, and wherein the first lens component is movable relative to the second lens component to focus the first objective lens assembly.

14. The stereoscopic endoscope of claim 13 further comprising:
an actuator system, wherein the actuator system is configured to receive first control signals to move the first lens component relative to the second lens component to focus the first objective lens assembly.

15. The stereoscopic endoscope of claim 14 wherein the actuator system is configured to receive second control signals to focus the second objective lens assembly independently of the first objective lens assembly.

16. The stereoscopic endoscope of claim 1 wherein the first objective lens assembly has a first focal plane and the second objective lens assembly has a second focal plane, and wherein the first focal plane and the second focal plane are approximately coincident.

17. The stereoscopic endoscope of claim 1 wherein the stereoscopic endoscope has a working distance from a distal end of the stereoscopic endoscope at which a target is located and wherein the first distal optical axis and the second distal optical axis converge at the working distance.

18. The stereoscopic endoscope of claim 1 wherein the first distal optical axis forms a first convergence angle with a longitudinal axis of the stereoscopic endoscope and the second distal optical axis forms a second convergence angle with the longitudinal axis and wherein the first convergence angle is approximately the same as the second convergence angle.

19. The stereoscopic endoscope of claim 1 wherein the first distal optical axis forms a first convergence angle with a longitudinal axis of the stereoscopic endoscope and the second distal optical axis forms a second convergence angle with the longitudinal axis and wherein the first convergence angle is different than the second convergence angle.

20. A method comprising:
directing a first light along a first distal optical axis through a first objective lens assembly;
after exiting the first objective lens assembly, changing a direction of at least a portion of the first light with an optical element to direct the at least a portion of the first light along a first proximal optical axis to the first surface of the first image capture sensor, wherein the first proximal optical axis is non-perpendicular to the first surface of the first image capture sensor;
directing a second light along a second distal optical axis through a second objective lens assembly; and
after exiting the second objective lens assembly, directing the second light along a second proximal optical axis to a second surface of a second image capture sensor, wherein the first distal optical axis is non-parallel to the second distal optical axis.

21. The method of claim 20 wherein the second proximal optical axis is non-perpendicular to the second surface of the second image capture sensor.

22. (canceled)

23. The method of claim
wherein the at least a portion of the first light includes a first portion of the first light and a second portion of the first light and wherein the optical element directs the first portion of the first light toward the first surface of the first image capture sensor and directs the second portion of the first light toward a third surface of a third image capture sensor.

24. The method of claim 20 further comprising:
focusing the first objective lens assembly by moving a first lens component of the first objective lens assembly relative to a second lens component of the first objective lens assembly.

25. The method of claim 24 wherein focusing the first objective lens assembly comprises
providing control signals to an actuator to move the first lens component relative to the second lens component.

26. The method of claim 24 further comprising:
focusing the second objective lens assembly by moving a first lens component of the second objective lens assembly relative to a second lens component of the second objective lens assembly, wherein the focusing of the first and second objective lens assemblies are controlled independently.
